(19) United States
(12) Patent Application Publication
(10) Pub. No.: US 2004/0014187 A1
(43) Pub. Date: Jan. 22, 2004

(54) Bacterial carboxypeptidase cpg2 variants and their use in gene directed enzyme prodrug therapy

(76) Inventors: Caroline Joy Springer, Sutton (GB); Richard Malcolm Marais, London (GB); Robert Spooner, Coventry (GB)

Correspondence Address: GRAY CARY WARE FREDENRICH, 1625 MASSACHUSETTS AVENUE, NW, SUITE 300, WASHINGTON, DC, 20036-2247, US

(21) Appl. No.: 10/275,580

(22) PCT Filed: May 4, 2001
(86) PCT No.: PCT/GB01/01988

(30) Foreign Application Priority Data
May 8, 2000 (GB) 0011060.1

(51) Int. Cl. C12N 9/48

(52) U.S. Cl. 435/170; 424/94.1; 435/183; 435/212

(57) ABSTRACT

The present invention relates to bacterial carboxypeptidases for use in gene directed prodrug therapy, in particular for use in the treatment of disease, including tumors. Specifically, the invention relates to modified bacterial carboxypeptidases which have enhanced catalytic activity.

### FIELD OF THE INVENTION

The present invention relates to gene directed enzyme prodrug therapy (GDEPT) and its use in treatment of disease, including tumours.

### BACKGROUND OF THE INVENTION

Gene-directed enzyme prodrug therapy (GDEPT) and virally directed enzyme prodrug therapies (VDEPT (Huber et al., 1994, Proc. Natl. Acad. Sci. 91, 8302-8306)) are suicide-gene therapy approaches that aim to increase the delivery of toxic metabolites to solid tumours. This aims to overcome one of the major problems associated with current therapies for cancer, i.e., the lack of specificity, resulting in harmful side effects to normal tissues, such as the gut lining and bone marrow. The term “GDEPT” is used to include both the viral and non-viral delivery systems.

In the first step, a gene encoding a foreign, prodrug-activating enzyme is delivered to tumour cells in such a fashion as to ensure its tumour-restricted expression. Subsequent systemic administration of an appropriate prodrug results in generation of toxic metabolites only at the tumour site and consequently tumour selective killing. A number of methods for ensuring tumour specific expression of the activating enzymes have been proposed, including injection of naked DNA (Vile et al. (1993) Cancer Res. 53; 3806-3864), targeted liposomes (Nabe et. al. (1994) Hum. Gene. Ther. 5,57-77), viruses (Hughes et al (1995) Cancer Res. 55; 3339-3345) and transcriptional regulation (Hughes et al (1995) Cancer Res. 55, 3339-3345; Ido et al. (1995) Cancer Res. 55, 3105-3109; Trinh et al. (1995) Cancer Res. 55, 4808-4812; Niculescu-Duvaz et al (1998) Bioconjugate Chemistry. 9, 4-22).

Our GDEPT system (WO 96/40238) focuses on the use of the enzyme carboxypeptidase G2 (CPG2), from Pseudomonas strain RS16. CPG2 activates benzoic acid mustard prodrugs such as 4-([2-chloroethyl] [2-mesyloxyethyl]amino)benzoyl-L-glutamic acid (CMDA) to release L-glutamic acid and the DNA alkylating drug 4-([2-chloroethyl] [2-mesoxyethyl]amino)benzoic acid, a potent cytotoxic agent (Springer et al. (1990) J. Med. Chem. 33, 677-681; Du Frain et al (1979) Envir. Mutagenesis. 1, 283-289; Frei et al (1988) Cancer Res. 48, 6427-6423; Teicher et al. (1988) Cancer Chemotherapy and Pharmacology 1988. 21: 292-298). CPG2 has a number of advantages over other enzyme/prodrug combinations. Unlike other systems, the toxic drug metabolite is released directly from the prodrug, without requiring further modification by cellular enzymes thus reducing the likelihood of induced drug resistance. Furthermore, the activated drugs are toxic to both cycling and non-cycling cells, a distinct advantage for chemotherapeutic agents.

Another advantage of CPG2 is that the enzyme does not require a co-substrate to activate the prodrug and therefore does not rely on cellular factors for activity. CPG2 is normally resident in the bacterial periplasm and is active as a homodimer (Sherwood et al. (1985) Eur. Biochem. 148, 447-453). The X-ray crystal structure of the protein reveals that each homodimer forms a dumbbell-like structure, comprising two distinct catalytic domains separated by the dimer interface (Rowsell et al. (1997) Structure, 5, 337-347). Association of amino acids from the N-(amino acids 22-213) and C-(amino acids 326-415) termini of the same monomer create each catalytic domain. The dimerisation domains are composed of a 110 amino acid (residues 214-325) insert between the two parts of the catalytic domain that folds into a four-stranded antiparallel β-sheet, flanked on one side by two α-helices. The dimer interface is stabilised by hydrophobic interactions between the helices and through hydrogen bonding between a β-strand from each monomer, forming a continuous β-sheet sheet across the dimer. This simple structure makes CPG2 highly versatile and active enzyme CPG2 has been expressed both within mammalian cells and tethered to their outer surfaces (Marais et al. (1996) Cancer Res. 56, 4735-4742; Marais et al. (1997) Nature Biotech. (1997) 15, 1373-1377).

Tethering to the outer surface of the cells was achieved by fusing a mammalian secretion signal to the N-terminus of CPG2 and a receptor tyrosine kinase transmembrane domain to its C-terminus, to act as a membrane anchor. Thus CPG2 was transported through the Golgi/endoplasmic reticulum and inserted into the outer side of the plasma membrane and this form of CPG2 is referred to as surface-tethered CPG2 (stCPG2). However, stCPG2 was inappropriately glycosylated on three asparigine residues (N222, N264, N272) which resulted in reduction in enzymatic activity. Some activity was restored by mutating these residues to glutamine to prevent glycosylation (referred to as stCPG2(Q)3).

### SUMMARY OF THE INVENTION

The present invention relates to the further mutation of these asparagine residues which resulted in improved enzymic activity.

Mutation of these residues showed that the asparagine at position 264 (N264) was an important amino acid for maintaining dimer stability, whereas mutation of the asparagines at positions 222 and 272 (N222 and N272) has a less severe effect on dimer stability.

The glutamine at position 264 in CPG2*(Q)3 was substituted with serine, threonine or alanine and dimer stability and enzyme activity were examined. Dimer stability was improved by the serine (CPG2*(QSQ)) substitution, whereas either the threonine (CPG2*(QTQ)) or alanine (CPG2*(QAQ)) did not restore dimer stability.

CPG2*(QSQ) is almost twice as active as CPG2*(Q)3, but its apparent affinity for MTX was decreased by almost 6-fold (Table 1). Furthermore, although CPG2*(QTQ) dimer stability was not improved, its catalytic activity was increased by ^{˜}2.5 fold, but it had a reduced apparent affinity for substrate (its Km was also increased by ^{˜}12 fold) compared to CPG2*(Q)3.

The invention provides a bacterial carboxypeptidase which, in its native form, comprises one or more asparagine residues, the residues being part of motifs which on expression in a mammalian cell are subject to N-linked glycosylation, wherein at least one Asn residue site is altered to serine, and which retains carboxypeptidase activity.

The preferred bacterial carboxypeptidase is a bacterial carboxypeptidase, which in its native form, comprises three Asn residues, Asn (1); Asn (2); Asn (3) numbered in the N terminal to C-terminal direction, the residues being part of motifs which on expression in a mammalian cell are subject to N-linked glycosylation, wherein Asn (2) is altered to serine.

Other alterations to Asn (2) may be made provided that the rate of conversion of enzyme to prodrug is substantially the same as that of the unchanged unglycosylated enzyme, as defined below. For example, in an alternative embodiment Asn (2) is changed to threonine.

Preferably Asn (1) and Asn (3) are also altered to amino acids other than asparagine, it is preferred that Asn (1) and/or Asn (2) are altered to glutamine.

In a preferred embodiment, the invention provides a bacterial carboxypeptidase CPG2 wherein residues Asn (1) and Asn (3) have each been altered to glutamine and Asn (2) has been altered to serine.

The preferred bacterial carboxypeptidase is the Pseudomonas carboxypeptidase CPG2 having the sequence shown in SEQ ID NO:2, wherein Asn (1) is at position 222, Asn (2) is at position 264, and Asn (3) is at position 272. It is preferred that Asn 264 is altered to serine. Asn 222 and Asn 272 may be altered to amino acids other than asparagine, preferably glutamine. In a preferred combination Asn 222 is altered to glutamine, Asn 264 is altered to serine and Asn 272 is altered to glutamine.

In an alternative embodiment Asn 264 is altered to threonine. Asn 222 and Asn 272 may be altered to amino acids other than asparagine, preferably glutamine. In a preferred combination Asn 222 is altered to glutamine, Asn 264 is altered to threonine and Asn 272 is altered to glutamine.

The invention further provides a vector comprising a nucleic acid sequence encoding said bacterial carboxypeptidase, optionally including a signal sequence capable of targetting the carboxypeptidase to the surface of a mammmalian cell. It is preferred that the signal sequence is a signal peptide of a transmembrane receptor kinase.

In another aspect the invention provides a two component system for use in association with one another comprising:

(a) a vector capable of expressing bacterial carboxypeptidase, wherein at least one Asn is altered to serine; and

(b) a pro-drug which can be converted into an active drug by said enzyme.

The bacterial carboxypeptidase may be located either in the cytosol or targetted to the surface of a cell. In a preferred embodiment the carboxypeptidase is expressed at the surface of a cell.

Preferred enzymes for use in the two component system of the invention are as described above.

The invention also provides for use of the altered bacterial carboxypeptidase CPG2, or the two component system in a method of treatment of a patient, a method of treating a tumour in a patient in need of treatment, or use of said carboxypeptidase CPG2 or vector encoding said carboxypeptidase in the manufacture of a medicament for the treatment of tumours.

The invention further provides a method of removing a —NH—CH(CO₂H)(Z) moiety from a compound to which the moiety is attached via an amide linkage, the method comprising contacting said compound with an enzyme described herein to effect removal of said moiety.

### BRIEF DESCRIPTION OF THE DRAWINGS

FIG. 1 shows gel electrophoresis monitoring of CPG2 dimer stability.

(A) shows detergent soluble extracts of COS cells transiently expressing β-gal, CPG2* or stCPG2(Q)3 were electrophoresed at 40° C. in non reducing conditions, either preheated or not heated. The positions of migration of dimers (d) and monomers (m) are indicated (arrowheads) as are the positions of molecular weight markers (×10⁻³).

(B) shows detergent soluble extracts of COS cells transiently expressing CPG2* and three CPG2* variants with N to L substitutions electrophoresed in the same conditions as A.

(C) shows non-heated extracts of COS cells transiently expressing CPG2* and substituted CPG2 variants, and stCPG2*(Q)3 and stCPG2 variants electrophoresed as in A.

FIG. 2 shows a time course of treatment with CMDA. WiDr cells (A) or mixtures of WiDR cells expressing β-gal containing 20% WiDR activator cells(B) and SK-OV-3 cells (C) or mixtures of SK-OV-3 cells expressing β-gal containing 20% SK-OV-3 activator cells (D) were treated with CMDA for the times indicated. Activator cells expressed CPG2* (open circles) or stCPG2(Q)3 (filled circles).

### DESCRIPTION OF THE SEQUENCE LISTING

SEQ ID NOs:1 and 2 show the nucleic acid amino acid sequences of carboxypeptidase CPG2 from Pseudomonas.

SEQ ID NOs: 3-6 show synthetic oligonucleotides used to generate altered CPG2 gene sequences, as discussed in the accompanying examples.

### DETAILED DESCRIPTION OF THE INVENTION

A. Enzyme Systems

The preferred enzyme is carboxypeptidase CPG2 (disclosed in WO88/07378), having the sequence shown in SEQ ID NO:2. Although other carboxypeptidases may be used. When expressed in eukaryotic cells, this enzyme undergoes N-linked glycosylation in the Golgi apparatus and endoplasmic reticulum at motifs whose primary amino acid sequence is Asn-Xaa-Ser/Thr (where Xaa is any amino acid residue). This leads to a reduction in activity compared with the unglycosylated form of the enzyme.

There are three such motifs in CPG2, located at Asn 222, Asn 264 and Asn 272. Alteration of these sites may improve activity. It is preferred that at least one of the sites is altered to serine, preferably Asn 264. The preferred alteration of these sites is where Asn 222 and Asn 272 are altered to glutamine and Asn 264 is altered to serine. The resultant QSQ motif has high catalytic activity and low Kₘ. Alternatively, Asn 264 may be altered to threonine. This results in increased catalytic activity and a high Kₘ. As discussed elsewhere herein, a high Kₘ may not be a disadvantage, for example in conditions of high prodrug concentration.

Other alternations to the motifs are also possible, provided that the alteration is such that the enzyme retains its ability to convert a prodrug to an active drug with substantially the same catalytic activity as the unchanged, unglycosylated enzyme. In this context “unsubstantially the same” will be preferably from 10, 5, 2, 1.5 or 1.25 fold less to 2, 5, or 10 fold more. For example Asn 264 may be changed to residues other than serine or threonine, provided that the rate of conversion of prodrug to active drug is substantially the same.

Other bacterial carboxypeptidase enzymes may be used, e.g., CPG2 enzymes from other Pseudomonas species such as Pseudomonas aeruginosa, Pseudomonas cepacia, Pseudomonas fluorescens, Pseudomonas putida, Pseudomonas syringae, Pseudomonas savastanoi, which in the native form comprise three asparagine residues, Asn (1), Asn (2), Asn (3) numbered in the N-terminal to C-terminal direction, the residues being part of motifs which on expression in a mammalian cell are subject to N-linked glycosylation. In such enzymes Asn (1), Asn (2) and Asn(3) will be at positions homologous to Asn 222, Asn 264 and Asn 272, although they may have different positional numbering. However, Asn (1), Asn (2) and Asn (3) of these enzymes can readily be identified by persons skilled in the art, for example using sequence alignments to compare a sequence with the sequence shown in SEQ ID NO:2, and thereby identify the Asn residues which correspond to Asn 222, Asn 264 and Asn 272 of SEQ ID NO:2.

CPG2 enzymes from other species of Pseudomonas may be obtained by routine cloning methodology. For example, a library of cDNA from a Pseudomonas species may be made and probed with all or a portion of the sequence of SEQ ID NO:2 under conditions of medium to high stringency.

For example, hybridizations may be performed, according to the method of Sambrook et al. (below) using a hybridization solution comprising: 5×SSC (wherein ‘SSC’=0.15 M sodium chloride; 0.15 M sodium citrate; pH 7), 5×Denhardt's reagent, 0.5-1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

Clones identified as positive may be examined to identify open reading frames encoding homologues of the sequence shown in SEQ ID NO:2. It may be necessary to combine more than one clone to achieve a full length open reading frame, as would be understood by the person skilled in art. Clones may then be expressed in a heterologous expression system, e.g. in bacteria or yeast and the protein purified by techniques known in the art.

Suitable enzymes to which mutations according to the invention may be applied include carboxypeptidase enzymes which are mutants, variants, derivatives or alleles of the sequence shown in SEQ ID NO:2. A carboxypeptidase enzyme which is a variant, allele, derivative or mutant may have an amino acid sequence which differs from that given in SEQ ID NO:2 by one or more of addition, substitution, deletion and insertion of one or more amino acids, for example from 1 to 20, such as from 1 to 10, e.g., 1, 2, 3, 4, 5 or 6-10 substitutions deletions or insertions.

Preferred such carboxypeptidases will have one or more of the following properties: immunological cross-reactivity with an antibody reactive the polypeptide for which the sequence given in SEQ ID NO:2; sharing an epitope with the polypeptide for which the amino acid sequence is shown in SEQ ID NO:2 (as determined for example by immunological cross-reactivity between the two polypeptides); a biological activity which is inhibited by an antibody raised against the polypeptide whose sequence is shown in SEQ ID NO:2; ability to release L-glutamic acid from benzoic acid mustard prodrugs. Alteration of sequence may change the nature and/or level of activity and/or stability of the carboxypeptidase enzyme.

A polypeptide which is an amino acid sequence variant, allele, derivative or mutant of the amino acid sequence shown in SEQ ID NO:2 may comprise an amino acid sequence which shares greater than about 35% sequence identity with the sequence shown, greater than about 40%, greater than about 50%, greater than about 60%, greater than about 70%, greater than about 80%, greater than about 90% or greater than about 95%. The sequence may share greater than about 60% similarity, greater than about 70% similarity, greater than about 80% similarity or greater than about 90% similarity with the amino acid sequence shown in the relevant figure. Amino acid similarity is generally defined with reference to the algorithm GAP (Genetics Computer Group, Madison, Wis.) as noted above, or the TBLASTN program, of Altschul et al. (1990) J. Mol. Biol. 215: 403-10. Parameters employed are the default ones: for nucleotide sequences—Gap Weight 50, Length Weight 3, Average Match 10.000, Average Mismatch 0.000; for peptide sequences—Gap Weight 8, Length Weight 2, Average Match 2.912, Average Mismatch −2.003. Peptide similarity scores are taken from the BLOSUM62 matrix. Also useful is the TBLASTN program, of Altschul et al. (1990) J. Mol. Biol. 215: 403-10, or BestFit, which is part of the Wisconsin Package, Version 8, September 1994, (Genetics Computer Group, 575 Science Drive, Madison, Wis., USA, Wisconsin 53711). Sequence comparisons may be made using FASTA and FASTP (see Pearson & Lipman, 1988. Methods in Enzymology 183: 63-98). Parameters are preferably set, using the default matrix, as follows: Gapopen (penalty for the first residue in a gap): −12 for proteins/−16 for DNA; Gapext (penalty for additional residues in a gap): −2 for proteins/−4 for DNA; KTUP word length: 2 for proteins/6 for DNA.

Sequence comparison may be made over the full-length of the relevant sequence shown herein, or may more preferably be over a contiguous sequence of about or greater than about 20, 25, 30, 33, 40, 50, 67, 133, 167, 200, 233, 267, 300, 333, or more amino acids or nucleotide triplets, compared with the relevant amino acid sequence or nucleotide sequence as the case may be.

The enzymes of the invention may otherwise be altered by truncation, substitution, deletion or insertion as long as the enzyme retains its ability to convert an a prodrug to an active drug with substantially catalytic activity. For example, small truncations in the N- and/or C-terminal sequence may occur as a result of the manipulations required to produce a vector in which. a nucleic acid sequence encoding the enzyme is linked to various other signal sequences described herein. The activity of the altered enzyme may be measured in model systems such as those described in the examples.

In further aspects the invention provides a nucleic acid encoding such modified bacterial carboxypeptidases or vectors comprising such nucleic acid. The vector is preferably an expression vector, wherein said nucleic acid is operably linked to a promoter compatible with a host cell. The invention thus also provides a host cell which contains an expression vector of the invention. The host cell may be bacterial (e.g. E. coli), insect, yeast or mammmalian (e.g. hamster or human)

Host cells of the invention may be used in a method of making a carboxypeptidase enzyme of the invention as defined above which comprises culturing the host cell under conditions in which said enzyme or fragment thereof is expressed, and recovering the enzyme in substantially isolated form. The enzyme may be expressed as a fusion protein.

B. Vector Systems

The vector may be any DNA or RNA vector used in VDEPT or GDEPT therapies.

Examples of suitable vector systems include vectors based on the Molony murine leukaemia virus (Ram, Z et al., Cancer Research (1993) 53; 83-88; Dalton and Triesman, Cell (1992) 68; 597-612. These vectors contain the murine leukaemia virus (MLV) enhancer cloned upstream at a β-globin minimal promoter. The β-globin 5′ untranslated region up to the initiation ATG is supplied to direct efficient translation of the cloned protein. The initiator ATG straddles an NcoI restriction site and thus can be used to clone a protein coding sequence into the vector. This vector further contains a polylinker to facilitate cloning, followed by the β-globin 5′ untranslated region and polyadenylation sites. The MLV enhancer is of particular use since it is a strong enhancer and is active in most murine and human cells.

Suitable viral vectors further include those which are based upon a retrovirus. Such vectors are widely available in the art. Huber et al., (Proc. Natl. Acad. Sci. USA (1991) 88, 8039) report the use of amphotropic retroviruses for the transformation of heptoma, breast, colon or skin cells. Culver et al (Science (1992) 256; 1550-1552) also describe the use of retroviral vectors in GDEPT. Such vectors or vectors derived from such vectors may also be used. Other retroviruses may also be used to make vectors suitable for use in the present invention. Such retroviruses include rous sarcoma virus (RSV). The promoters from such viruses may be used in vectors in a manner analagous to that described above for MLV.

Englehardt: et al (Nature Genetics (1993)4; 27-34) describes the use of adenovirus based vectors in the delivery of the cystic fibrosis transmembrane conductance product (CFTR) into cells, and such adenovirus based vectors may also be used. Vectors utilising the adenovirus promoter and other control sequences may be of use in delivering a system according to the invention to cells, in particular the cells of the lung, and hence useful in treating lung tumours.

C. Other Vector Components

In system according to the invention the enzyme may be linked to a signal sequence which directs the enzyme to the surface of a mammalian cell. This will be needed unless the enzyme has an endogenous signal which does this. Even if an enzyme does have such a signal sequence, it can be replaced where this is desirable or appropriate. Suitable signal sequences include those found in transmembrane receptor kinases such as the c-erbB2 (HER2/neu) signal sequence or variants thereof which retain the ability to direct expression of the enzyme at the cell surface. The c-erbB2 signal sequence can be obtained by reference to Coussens et al (1985) Science 230; 1132-1139.

Variants of the signal sequence may be produced using standard techniques known as such in molecular biology, e.g. site directed mutagenesis of a vector containing the signal sequence.

Further suitable signal sequences include those disclosed in von Heijne (1985) J. Mol. Biol. 184; 99-105.

The enzyme of the invention may be expressed at the surface of the cell. In this case it is expressed in such a way as to expose the enzyme outside the cell so that it may interact with the prodrug, but will still be attached to the plasma membrane by virtue of a suitable plasma membrane anchor. A suitable anchor will be a polypeptide anchor which is expressed by the vector. For example, the enzyme may be linked to a sequence which is a transmembrane region which anchors the enzyme in the membrane of the cell. Such a transmembrane region can be derived from transmembrane receptor kinases, such as c-erbB2, EGF receptors and CSF-1 receptors. The c-erbB2 transmembrane region is set out in the example below. Variants of such transmembrane regions may also be used provided they retain the ability to anchor the enzyme in the membrane of a cell, such that the active portion of the enzyme is outside the cell, and at its surface. Other anchors e.g., peptidoglycan anchors are lipid anchors and could also be used.

The anchor such as the one from the transmembrane region is attached to the open reading frame of the open reading frame of the enzyme gene by suitable molecular biology techniques. When the protein is expressed it will have the anchor attached as the enzyme anchor fusion protein is made. The anchor will then be embedded in the membrane and will hold the enzyme there.

Vectors encoding the enzyme, together with, when required, a signal sequence and/or transmembrane region may be made using recombinant DNA techniques known in the art. The sequence encoding the enzyme, signal sequence and transmembrane regions may be constructed by splicing synthetic or recombinant nucleic acid sequences together, or modifying existing sequences by techniques such as site directed mutagenesis. Reference may be made to “Molecular Cloning” by Sambrook et al (1989), Cold Spring Harbour) for discussion of standard recombinant DNA techniques.

D. Promoters

The enzyme will be expressed in the vector using a promoter capable of being expressed in the cell to which the vector is targeted. The promoter will be operably linked to the sequences encoding the enzyme and its associated sequences. For example the promoter may be the c-erbB2 promoter. The c-erbB2 proto-oncogene (Hudson et al, (1990) J. Biol. Chem. 265; 4389-4393) is expressed in breast tissue at low levels and in a tissue restricted manner. In some tumour states however the expression of this protein is increased due to enhanced transcriptional activity. Notable examples of this are breast tissue (about 30% of tumours), ovarian (about 20%) and pancreatic tumours (about 50-75%). In such tumours where expression of c-erbB2 is increased due to enhanced transcription or translation, the c-erbB2 promoter may be used to direct expression of proteins in a cell specific manner.

Utilising the c-erbB2 promoter with the GDEPT system of the present invention to target such tumours increase the specificity of GDEPT, since transfection of normal cells with a c-erbB2 promoter will provide only limited amount of enzyme expression and this limited activation of prodrug.

In general, those of skill in the art will appreciate that some regions of the promoter such as those at -213 will need to be retained to ensure tumour specificity of expression from the vector whereas other regions of the promoter may be modified or deleted without significant loss of specificity. Thus, modified promoters which are transcriptionally regulated substantially to the same degree as human c-erbB2 are preferred. The degree of regulation of such candidate promoters can be tested and assessed by those of skill in the art using for example CAT assays in accordance with those described in Hollywood and Hurst. “Operably linked” means joined as part of the same nucleic acid molecule, suitably positioned and oriented for transcription to be initiated from the promoter. DNA operably linked to a promoter is “under transcriptional initiation regulation” of the promoter. Thus there may be elements such as 5′ non-coding sequence between the promoter and coding sequence which is not native to either the promoter nor the coding sequence. Such sequences can be included in the vector if they do not impair the correct control of the coding sequence by the promoter.

Other suitable promoters include viral promoters such as mammalian retrovirus or DNA virus promoters. Suitable promoters include those used in vectors described above, e.g. MLV, CMV, RSV and adenovirus promoters. Preferred adenovirus promoters are early gene promoters. Strong mammmalian promoters may also be suitable. An example of such a promoter is the EF-1α promoter which may be obtained by reference to Mizushima and Nagata (1990) Nucl. Acids Res. 18; 5322. Variants of such promoters retaining substantially similar transcriptional activities may also be used.

E. Prodrugs

The prodrug for use in the system will be selected to be compatible with the CPG2 carboxypeptidase such that the enzyme will be capable of converting the prodrug to the active drug. Desirably, the toxicity of the prodrug to the patient being treated will be at least one order of magnitude less toxic to the patient than the active drug. Preferably the active drug will be several, e.g. 2, 3 or 4 or more orders of magnitude more toxic than the prodrug. Nitrogen mustard prodrugs are preferred. Other suitable prodrugs include those disclosed in WO96/03515.

Nitrogen mustard prodrugs include compounds of the formula:

M-Ar-CONH-R

where Ar represents an optionally substituted ring aromatic ring system, R-NH is the residue of an α-amino acid R-NH₂ or oligopeptide R-NH₂ and contains at lease one carboxylic acid group, and M represents a nitrogen mustard group.

The residue of the amino acid R-NH is preferably the residue of glutamic acid. It is disclosed in WO88/07378 that the enzyme carboxypeptidase G2 is capable of removing the glutamic acid moiety from compounds of the type shown above, and the removal of the glutamic acid moiety results in the production of an active nitrogen mustard drug.

Thus nitrogen mustard prodrugs of use in the invention include the prodrugs of generic formula I of WO94/02450 and salts thereof, and in particular those of formula (I):

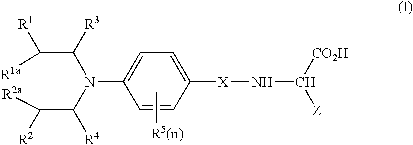

wherein R¹ and R² each independently represent chlorine, bromine, iodine, OSO₂Me, OSO₂phenyl (wherein phenyl is optionally substituted with 1,2,3,4 or 5 substituents independently selected from C₁₋₄ alkyl, halogen, —CN or NO₂);

R^{1a} and R^{2a} each independently represents hydrogen, C₁₋₄ alkyl or C₁₋₄ haloalkyl;

R³ and R⁴ each independently represents hydrogen, C₁₋₄ alkyl or C1-4 haloalkyl;

n is an integer from 0 to 4;

each R⁵ independently represents hydrogen, C₁₋₄ alkyl optionally containing one double bond or one triple bond, C₁₋₄ alkoxy, halogen, cyano, haloalkyl (e.g. —CF₃) —NH2, —CONR⁷R⁸ (wherein R⁷ and R⁸. are independently hydrogen, C₁₋₆ alkyl or C3-6 cycloalkyl) or two adjacent R⁵ groups together represent

a) C4 alkylene optionally having one double bond;

b) C3 alkylene; or

c) —CH═CH—CH═CH—, —CH—CH—CH2— or —CH2—CH═CH— each optionally substituted with 1,2,3 or 4 substituents each independently selected from the group consisting of C₁₋₄ alkyl, C₁₋₄ alkoxy, halogen, cyano or nitro;

X is a group —C(O)—, —O—C(O)—, —NH—C(O)— or —CH2—C(O); and

Z is a group —CH₂—T—C(O)—OR⁶ where T is CH₂, —O—, —S—, —(SO)— or —(SO2)—, and R⁶ is hydrogen, C₁₋₆ alkyl, C₃₋₆ cycloalkyl amino, mono- di-C₁₋₆ alkylamino or mono or diC₃₋₆ cycloalkyl amino, provided that when R⁶ is hydrogen T is —CH2—; and physiologically acceptable derivatives, including salts, of the compounds of formula (I).

Halogen includes fluorine, chlorine, bromine and iodine. Preferred values for the groups R^{1a} and R^{2a} are methyl and hydrogen, especially hydrogen. Preferred values for the groups R³ and R⁴ are hydrogen, methyl and trifluoromethyl, especially hydrogen. Preferred values for the groups R¹ and R² and I, Br, Cl, OSO₂Me and OSO₂phenyl wherein phenyl is substituted with one or two substituents in the 2 and/or 4 positions. I, Cl and OSO₂Me are especially preferred.

Preferred values for R⁵ when n is an integer from 1 to 4 are fluorine, chlorine, methyl-CONH₂ and cyano. Preferably, n is 0, 1 or 2. When n is 1 or 2 it is preferred that R⁵ is fluorine at the 3 and/or 5 positions of the ring. The group X is preferably —C(O)—, —O—C(O)— or —NH—C(O)—. Z is preferably a group —CH₂CH₂—COOH.

Preferred specific compounds include:

N-4-[(2-chloroethyl) (2-mesyloxyethyl) amino]benzoyl-L-glutamic acid (referred to below as “CMDA”) and salts thereof;

N-(4-[bis(2-chloroethyl)amino]-3-fluorophenylcarbamoyl)-L-glutamic acid and salts thereof;

N-(4-[bis(2-chloroethyl)amino]phenylcarbamoyl)-L-glutamic acid and salts thereof;

N-(4-[bis(2-chloroethyl)amino]phenoxycarbonyl)-L-glutamic acid and salts thereof;

N-(4-[bis(2-iodoethyl)amino]phenoxycarbonyl)-L-glutamic acid (referred to below as “prodrug 2”) and salts thereof;

N-(3,5-difluoro-4-[bis(2-iodoethyl)amino]phenoxycarbonyl)-L-glutamic acid, and salts thereof;

N-(3,5-difluoro-4-[bis(2-chloroethyl)amino]benzoyl)-L-glutamic acid, and salts thereof;

N-(3,5-difluoro-4-[bis(2-bromoethyl)amino]benzoyl)-L-glutamic acid, and salts thereof;

N-(2,3,5-trifluoro-4-[bis(2-chloroethyl)amino]benzoyl)-L-glutamic acid;

N-(2,3,5-trifluoro-4-[bis(2-bromoethyl)amino]benzoyl)-L-glutamic acid, and salts thereof;

N-(2,3,5-trifluoro-4-[bis(2-iodoethyl)amino]benzoyl)-L-glutamic acid, and salts thereof;

N-(3,5-difluoro-4-[bis(2-bromopropyl)amino]benzoyl)-L-glutamic acid, and salts thereof;

N-(3-trifluoromethyl-4-[bis(2-bromoethyl)amino]benzoyl)-L-glutamic acid, and salts thereof.

Particular sub-groups of the compounds of the present invention of interest may be obtained by taking any one of the above mentioned particular or generic definitions for R¹-R⁴, R⁵, X or W either singly or in combination with any other particular or generic definition for R¹-R⁴, R⁵, X or W.

Derivatives

Physiologically acceptable derivatives of prodrugs include salts, amides, esters and salts of esters. Esters include carboxylic acid esters in which the non-carbonyl moiety of the ester grouping is selected from straight or branched chain C₁₋₆ alkyl, (methyl, n-propyl, n-butyl or t-butyl); or C₃₋₆ cyclic alkyl (e.g. cyclohexyl). Salts include physiologically acceptable base salts, e.g. derived from an appropriate base, such as alkali metal (e.g. sodium), alkaline earth metal (e.g. magnesium) salts, ammonium and NR₄″ (wherein R″ is C₁₋₄ alkyl) salts. Other salts include acid addition salts, including the hydrochloride and acetate salts. Amides include non-substituted and mono- and di-substituted derivatives.

F. Applications of the Invention

The system of the invention can be used in a method of treatment of the human or animal body. Thus the two component system may be supplied as the two products (enzyme or microbe, plus prodrug) in the form of a kit, optionally with instructions for use of the two products. The two components may be provided separately to the vicinity of a patient, and brought together for sequential administration to such a patient.

Treatment in accordance with the invention includes a method of treating the growth of neoplastic cells which comprises administering to a patient in need of treatment the system of the invention. It is also possible that the invention may be used to treat cells which are diseased through infection of the human or animal body by bacteria, viruses or parasites. Viral late promoters often rely on viral proteins that are made early in the infection. The viral coat proteins which are expressed on the surface of an infected cell may be used as a target for getting the gene into the cell. If a viral late promoter is then used to direct expression of the GDEPT enzyme, any infected cells will express the protein, and specifically, cells which have been infected, for some time. This may be sufficient to kill the infected cells. For parasites, a parasite promoter and parasite surface proteins, may be used to direct expression and infect the parasites respectively.

For a bacteria, all the delivery systems will probably need to be changed to use bacterial viruses, although a specific promoter should be easier to define.

For use of the vectors in therapy, the vectors will usually be packaged into viral particles and the particles delivered to the site of the tumour, as described in for example Ram et al (ibid). The viral particles may be modified to include an antibody, fragment thereof (including a single chain) or tumour-directed ligand to enhance targeting of the tumour. Alternatively, the vectors may be packaged into liposomes. The liposomes may be targeted to a particular tumour. This can be achieved by attaching a tumour-directed antibody to the liposomes. Viral particles may also be incorporated into liposomes. The particles may be delivered to the tumour by any suitable means at the disposal of the physician. Preferably, the viral particles will be capable of selectively infecting the tumour cells. By “selectively infecting” it is meant that the viral particles will primarily infect tumour cells and that the proportion of non-tumour cells infected is such that the damage to non-tumour cells by administration of a prodrug will be acceptably low, given the nature of the disease being treated. Ultimately, this will be determined by the physician.

One suitable route of administration is by injection of the particles in a sterile solution. While it is possible for the prodrugs to be administered alone it is preferable to present them as pharmaceutical formulations. The formulations comprise a prodrug, together with one or more acceptable carriers thereof and optionally other therapeutic ingredients. The carrier or carriers must be “acceptable” in the sense of being compatible with the other ingredients of the formulation and not deleterious to the recipients thereof, for example, liposomes. Suitable liposomes include, for example, those comprising the positively charged lipid (N[1-(2,3-dioleyloxy)propyl]-N,N,N-triethylammonium (DOTMA), those comprising dioleoylphosphatidylethanolamine (DOPE), and those comprising 3α[N-(n′,N′-dimethylaminoethane)-carbamoyl] cholesterol (DC-Chol).

Viruses, for example isolated from packaging cell lines may also be administered by regional perfusion or direct intratumoral direction, or direct injection into a body cavity (intracaviterial administration), for example by intra-peritoneum injection.

It is also known that muscle cells can take up naked DNA and thus sarcomas may be treated using a vector system of the invention in which naked DNA is directly injected into the sarcoma.

Formulations suitable for parenteral or intramuscular administration include aqueous and non-aqueous sterile injection solutions which may contain anti-oxidants, buffers, bacteriostats, bactericidal antibiotics and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. The formulations may be presented in unit-dose or multi-dose containers, for example sealed ampoules and vials, and may be stored in a freeze-dried (lyophilized) condition requiring only the addition of the sterile liquid carrier, for example water, for injections, immediately prior to use. Injection solutions and suspensions may be prepared extemporaneously from sterile powders, granules and tablets of the kind previously described.

It should be understood that in addition to the ingredients particularly mentioned above the formulations may include other agents conventional in the art having regard to the type of formulation in question. Of the possible formulations, sterile pyrogen-free aqueous and non-aqueous solutions are preferred.

The doses may be administered sequentially, eg. at daily, weekly or monthly intervals, or in response to a specific need of the patient. Preferred routes of administration are oral delivery and injection, typically parenteral or intramuscular injection or intratumoural injection.

In using the system of the present invention the prodrug will usually be administered following administration of the vector encoding an enzyme. Typically, the vector will be administered to the patient and then the uptake of the vector by transfected or infected (in the case of viral vectors) cells monitored, for example by recovery and analysis of a biopsy sample of targeted tissue.

The exact dosage regime will, of course, need to be determined by individual clinicians for individual patients and this, in turn, will be controlled by the exact nature of the prodrug and the cytotoxic agent to be released from the prodrug but some general guidance can be given. Chemotherapy of this type will normally involve parenteral administration of both the prodrug and modified virus and administration by the intravenous route is frequently found to be the most practical. For glioblastoma the route is often intratumoural. A typical dosage range of prodrug generally will be in the range of from about 1 to 150 mg per kg per patient per day, which may be administered in single or multiple doses. Preferably the dose range will be in the range from about 10 to 75, e.g. from about 10 to 40, mg per kg per patient per day. Other doses may be used according to the condition of the patient and other factors as the discretion of the physician.

Tumours which may be treated using the system of the present invention include any tumours capable or being treated by a GDEPT or VDEPT system and thus are not limited to any one particular class of tumours. Particularly suitable tumour types include breast, colorectal and ovarian tumours, as well as pancreatic, melanoma, glioblastoma, hepatoma, small cell lung, non-small cell lung, muscle and prostate tumours.

The system or enzyme of the invention may be alternatively used in conjunction with the bacterial delivery systems. For example, WO 96/40238 describes a method wherein genes are delivered to tumour cells by genetically engineered, tumour-specific micro-organisms. Salmonella species of bacteria, or Mycobacterim avium, or by the protozoan Leishmania amazonensis are preferred micro-organisms for use as delivery systems, as each shows natural preference for attachment to and penetration into tumour cells, as opposed to non-cancerous cells. Prodrug converting enzymes can be expressed in these bacteria and targetted to the tumour. The modified CPG2 of the invention could be expressed in such a bacterial system and thereby targetted to the tumour cells.

The system of the invention may also be used to treat infections diseases, for example, and any other condition which requires eradication of the population of cells.

It will be understood that where treatment of tumours is concerned, treatment includes any measure taken by the physician to alleviate the effect of the tumour on a patient. Thus, although complete remission of the tumour is a desirable goal, effective treatment will also include any measures capable of achieving partial remission of the tumour as well as a slowing down in the rate of growth of a tumour including metastases. Such measures can be effective in prolonging and/or enhancing the quality of life and relieving the symptoms of the disease.

The invention will now be described in detail with reference the following examples.

### EXAMPLES

### Materials and Methods

(1) Preparation of Mutants

The plasmids pMCEFcpg2*, pMCEFcpg2(Q)3 and pMCEFstcpg2(Q)3, encoding respectively cytosolic CPG2*, cytosolic CPG2* bearing three asparagine to glutamine mutations, and surface tethered stCPG2(Q)3 with the same three mutations have been described (Marais et al (1996) Cancer Res. 56, 4735-4742; Marais et al (1997) Nature Biotech. 15, 1373-1377).

A BsmFI recognition site was inserted downstream of the codon expressing CPG2 amino acid S274 in pEFcpg2(Q)3 as follows. Two PCR fragments generated using a primer recognising the 5′ end of the CPG2 gene and oligonucleotide 1, and a primer recognising the 3′ end of the CPG2 gene and oligonucleotide 2, were fused by mixing the fragments and amplifying using the flanking 5′ and 3′ primers. An internal SphI-SalI fragment from this fusion was used to replace the corresponding region in pEFcpg2*(Q)3, generating a plasmid in which the CPG2*(Q)3 coding sequence was interrupted by introduction of the BsmFI sequence GGGAC. A further BsmFI recognition site was introduced into this plasmid using a similar strategem, but replacing oligonucleotides 1 and 2 with oligonucleotides 3 and 4. This generated a plasmid in which the CPG2* coding sequence is interrupted downstream of the codon encoding S274 with the BsmFI sequence GGGAC, and upstream of the codon encoding N264 with GTCCC, the BsmFI sequence in the opposite orientation.

| | | |
|---|---|---|
| Oligonucleotide 1: | CGCCAAGGCCGGCCAAGTCTCGGGGACAACATCATCCCCGCC | (SEQ ID NO:3) |
| Oligonucleotide 2: | GGCGGGGATGATGTTGTCCCCGAGACTTGGCCGGCCTTGGCG | (SEQ ID NO:4) |
| Oligonucleotide 3: | AAGAAACCTGCGCTTCGTCCCCAATGGACCATCGCC | (SEQ ID NO:5) |
| Oligonucleotide 4: | GGCGATGGTCCATTGGGGACGAAGCGCAGGTTCTT | (SEQ ID NO:6) |

The gene containing the two BsmFI sites was fused to the c-erbB2 signal peptide and the c-erbB2-CPG2(Q)3 fusion gene containing the two BsmFI sites was subcloned into pUC18, which has no BsmFI sites, under the transcriptional control of the lac promoter. After cutting with BsmF1, this plasmid's large fragment was ligated to sets of hybridised oligonucleotides that replaced the missing section and permitted alteration of specific sites. After transformation, CPG2 genes encoding altered amino acids were identified by DNA sequencing and activity of the encoded enzymes was screened by examining the ability of conditioned growth medium to degrade MTX, a good CPG2 substrate. Altered genes were further subcloned to generate plasmids encoding CPG2* and stCPG2 variants under Ef1-a transcriptional control for transient expression in mammalian cells.

(2) Generation of Cell Lines Constitutively Expressing stCPG2(Q)3

All mammalian cell lines were maintained in Dulbecco's Modified Eagle's Medium supplemented with 10% foetal calf serum (DMEM/FCS). For this study, the human colorectal carcinoma cell line WiDr (Noguchi et al (1979) In Vitro. 15, 401-408, and the human ovarian carcinoma cell lines SK-OV-3 (Hill et al. (1987) 39, 219-225) and A2780 (Louie et al. (1985) Cancer Res. 45, 2110-2115) were separately transfected with the plasmid pMCEFstcpg2(Q)3 (Marais et al (1997) Nature Biotech. 15, 1373-1377) and G418-resistant colonies were selected by limiting dilution. Detergent soluble extracts were incubated with methotrexate, a good carboxypeptidase G2 substrate. Rate of change of absorbance at 320 nm was measured to identify those colonies able to degrade MTX and therefore likely to express stCPG2(Q)3. Expression of stCPG2(Q)3 was confirmed by immunoblotting these detergent soluble extracts using a rabbit polyclonal serum specific for CPG2 (Marais et al (1996) Cancer Res. 56, 4735-4742; Marais et al (1997) Nature Biotech. 15, 1373-1377). Cell lines constitutively expressing β-galactosidase or CPG2* have been described (Marais et al (1996) Cancer Res. 56, 4735-4742; Marais et al (1997) Nature Biotech. 15, 1373-1377).

(3) Enzyme Kinetic Studies

COS-7 cells were used for transient expression to provide detergent soluble extracts containing large amounts of CPG2*, CPG2*(Q)3 and stCPG2(Q)3 enzymes for enzyme kinetic studies. Detergent soluble cell extracts, and enzyme kinetic analyses have been described previously (Marais et al (1996) Cancer Res. 56, 4735-4742; Marais et al (1997) Nature Biotech. 15, 1373-1377). Levels of CPG2 protein in detergent soluble extracts were determined by quantitative immunoprotein blotting using a PhosphorImager standardised with purified CPG2 expressed in insect cells. Kinetic parameters of the CPG2 derived proteins were measured, in all cases using 50 ng of CPG2* per assay, and the equivalent amount of the internally expressed and cell-surface tethered variants. The specific activity of COS/CPG2* preparations containing 50 ng of CPG2* protein was assigned as 100%.

(4) Kinetics of Cell Death and Cytotoxicity Assays

To determine exposure times to CMDA required for the death of 50% of the cell populations, cell lines were plated at 3×10⁵ cells per well in 6-well tissue culture plates, and allowed to grow to confluence. Tissue culture medium was replaced with 1 ml DMEM/FCS containing 2 mM CMDA (for WiDr and SK-OV-3 cell lines) or 1 mM CMDA (for A2780 cell lines). These concentrations are not toxic to control cell lines expressing lacZ. After 0.5, 1, 2, 4, 8, 12, and 16 h exposure, cells were trypsinized and approximately 3% were replated. After a further 4 d growth, cell survival was determined by [³H]-thymidine incorporation (0.4 μCi/ml, 6h).

Cytotoxicity and bystander cytotoxicity assays were performed as described previously (Marais et al (1996) Cancer Res. 56, 4735-4742). Briefly, for cytotoxicity assays, cells were grown to confluence and treated twice, once for an hour, immediately followed by an 18 h treatment, using increasing amounts of CMDA prodrug in DMEM/FCS. Cells were trypsinized and approximately 3% were replated and allowed to grow for a further 4 days, when uptake of [³H]-thymidine was used to infer survival. For bystander assays, mixtures of cells expressing stCPG2(Q)3 and cells of the same lineage expressing β-galactosidase were treated with a single concentration of CMDA in the same two-stage treatment protocol, using 2 mM CMDA for WiDr and SK-OV-3 cells and 1 mM CMDA for A2780 cells. Under these conditions, these concentrations of CMDA do not kill the bystander recipient cell lines when tested in the absence of stCPG2 (Q)3 expressing cells.

(5) Synthesis of CMDA

CMDA prodrug was synthesised as described previously (Springer et al. (1993) 18, 212-215.

### Example 1

Characterisation of Surface Tethered CPG2

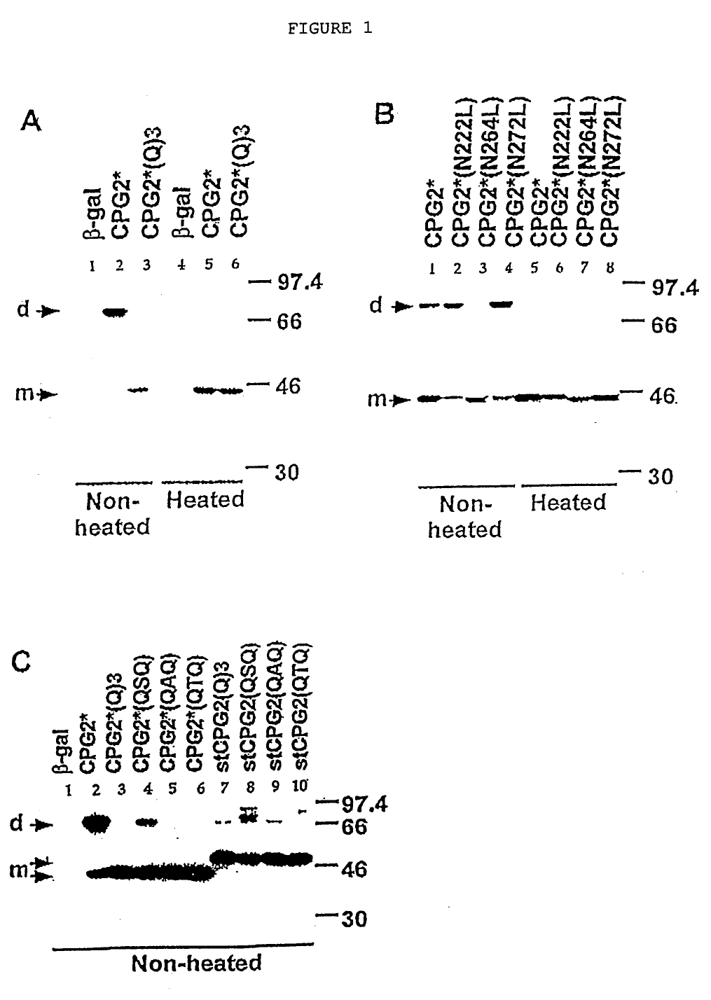

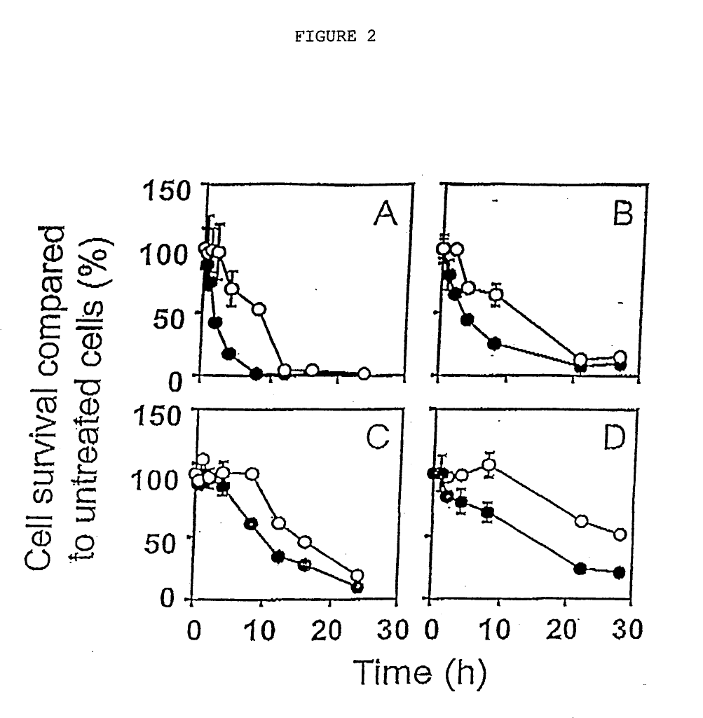

We first determined why the asparagine to glutamine mutations that blocked glycosylation reduced CPG2s enzyme activity. By examining the crystal structure of CPG2, we observed that all three amino acids lie within the dimerisation domain of CPG2, suggesting that the loss of activity could have been due to effects on dimer stability. To test this, CPG2* was transiently expressed in COS cells and the stability of the dimers determined by non-reducing SDS-polyacrylamide gels. CPG2 dimers are highly stable, and in this gel system, the CPG2* dimer migrated with an apparent Mr of ^{˜}80,000 (FIG. 1A, lane 2). The dimers were destabilised by heating the sample prior to gel loading and the monomers migrated with an apparent Mr of ^{˜}42,000 (FIG. 1A lane 5). By contrast, dimers formed by CPG2* in which N222, N264 and N272 were all substituted with glutamines (CPG2*(Q)3) were unstable and migrated as monomers even when the sample was not heated (FIG. 1A lanes 3, 6). We next tested each position independently. CPG2*(N222L) and CPG2*(N272) both formed stable dimers in this gel system, whereas CPG2*(N264L) migrated as a monomer (FIG. 1 B lanes 2,3,4), suggesting that N264 was an important amino acid for maintaining dimer stability. This is consistent with its position within the dimerisation interface. The side chain of each N264 residue is buried within this interface, forming interactions with the opposite chain, whereas N222 and N272 are at the surface and do not form similar interactions, so that their mutation has a less severe effect on dimer stability.

The glutamine for asparagine substitution is not the most sterically favourable, since the glutamine side chain is considerably larger than that of asparagine. This mutation would thus cause some distortion of the dimer interface and so we tested whether substitution with amino acids with smaller side chains would result in improved dimer stability and enzyme activity. The glutamine at position 264 in CPG2*(Q)3 was substituted with serine, threonine or alanine and dimer stability and enzyme activity were examined. Dimer stability was not improved by either the threonine (CPG2*(QTQ)) or alanine (CPG2*(QAQ)) substitutions, whereas the serine (CPG2*(QSQ)) substitution restored weak dimer stability (FIG. 1 C, lanes 4,5, 6) This restoration of dimer stability had complex effects on enzyme activity. Although CPG2*(QSQ) is almost twice as active as CPG2*(Q)3, its apparent affinity for MTX was decreased by almost 6-fold (Table 1). Furthermore, although CPG2*(QTQ) dimer stability was not improved, its catalytic activity was increased by ^{˜}2.5 fold, but its Km was also increased by ^{˜}12 fold, compared to CPG2*(Q)3. CPG2*(QAQ) was essentially inactive.

We next investigated how these mutations affected stCPG2. As with CPG2*,the threonine (stCPG2(QTQ)) and the alanine (stCPG2(QAQ) substituted proteins did nor form stable dimers, whereas weak dimers were seen with the serine substituted protein (stCPG2(QSQ)) (FIG. 1 C, lanes 8,9,10). In fact, stCPG2 (QSQ) has kinetic properties which are comparable to that of stCPG2(Q)3. Intriguingly, despite not improving dimer stability, surface tethering did allow substantial recovery of activity in the alanine-substituted protein. Thus, whereas CPG2*(QAQ) was inactive, stCPG2(QAQ) had high enzyme activity (38% of CPG2*) and a Km for MTX of 69 μM. All four stCPG2 variants had Km values in the range of 45-69 μM MTX and catalytic activities of 29-40% of that of CPG2*. Even the low Km of CPG2* (Q) 3, was increased to 54 μM by the surface tethering process.

Taken together, these data demonstrate that N264 substitutions affect CPG2 dimer stability, enzyme activity and substrate affinity when the protein is expressed in the cytosol.

Mutations were also performed at threonine 266 (T₂₆₆), in the context of Q₂₂₂ and Q₂₇₂. While most alterations of this threonine residue resulted in an enzyme with very low activity or an inactive enzyme, alteration of T₂₆₆ to V₂₆₆ resulted in 55% activity and a Km of 90 μm.

### Example 2

Surface Tethered CPG2 Sensitises Mammalian Cells to CMDA

In order to conduct these studies, we engineered WiDr (human colorectal adenocarcinoma), SK-OV-3 and A2780 (both human ovarian tumour lines) cells for stable expression of stCPG2(Q)3. The presence of stCPG2(Q)3 was verified by immunoprotein blotting (data not shown) and the levels of enzyme activity in these clones were determined using MTX as substrate (Table 2). For each line two or three clones were examined. The SK-OV-3 clones expressed the lowest levels of CPG2 activity (0.016 and 0.023 U/mg protein) followed by the A2780 clones (0.06 and 0.067 U/mg), with WiDr clones expressing the highest level of CPG2 activity (0.177 and 0.226 U/mg) (Table 2).

All of these isolates were more sensitive than parental cells expressing β-galactosidase (β-gal) to the prodrug CMDA. The A2780 clones were the most sensitive with IC₅₀ values in the range of ^{˜}15-21 μM CMDA, an increase in sensitivity of >100 fold compared to β-gal expressing cells (IC₅₀ 2150 μM, Table 2). WiDr cell expressing stCPG2(Q)3 had IC₅₀ values in the range of 100-150 μM, between 22 and 32 fold more sensitive than the β-gal expressing WiDr cells (IC₅₀>3200, Table 2). Finally, SK-OV-3 clones expressing stCPG2(Q)3 had IC₅₀ values of 200 to 550 μM, an increased sensitivity of 7-20 fold compared to β-gal expressing controls (Table 2). These data show that all three cell lines could be rendered sensitive to CMDA by expression of stCPG2(Q)3, although the levels of enzyme activity could not be used as an indication of susceptibility. Thus, although the WiDr clones expressed ^{˜}3 times more enzyme activity than the A2780 clones, the A2780 clones were 5-10 times more sensitive to CMDA than the WiDr clones (Tables 1, 2). Similarly, although WiDr clones expressed ^{˜}10 times more enzyme activity than the SK-OV-3 clones, the WiDr clones were only 1.5-5.5 times more sensitive to CMDA than the SK-OV-3 clones (Table 1, 2).

It is clear that the levels of CPG2 activity in these stCPG2(Q)3 expressing clones were consistently lower than we have previously shown to be the case for CPG2* expressing clones and yet similar levels of susceptibility to CMDA were seen. For example, the A2780 clone expressing CPG2* from our previous study contained 0.964 U/mg CPG2 (Marais et al. (1996) Cancer Res. 56, 4735-4742), ^{˜}15 times more enzyme activity than the stCPG2(Q)3 expressing clones from this study (Table 2) and yet the CPG2* clone had an IC₅₀ for CMDA of 23.2 μM (ref), similar to the 15 to 2μM values reported here for the stCPG2(Q)3 clones (Table 2). Similarly, the WiDr clone expressing CPG2* contained 0.787 U/mg (Marais et al. (1996) Cancer Res. 56, 4735-4742) CPG2 enzyme activity, about 3.5 to 4.5 times more CPG2 activity than the stCPG2(Q)3 clones (Table 2), and yet its IC₅₀ of 277 μM was actually slightly higher than the IC₅₀ values reported for the CPG2* expressers (100/147μM). Finally, the SK-OV-3 clone expressing CPG2* contained 1.013 U/mg CPG2, about 44 to 63 times more enzyme activity than the stCPG2(Q)3 clones (Table 2), but its IC₅₀ for CMDA at 258 μM fell within the range of the enzyme activity seen with the stCPG2(Q)3 clones (216-544μM, Table 2).

### Summary of Examples

Surface tethering of CPG2(Q)3 in MDA MB 361 cells results in a reduction in enzyme activity, in part because the mutations that prevent glycosylation reduce enzyme activity (Marais et al. (1997) Biotechnology 15, 1373-1377). Since the glycosylated residues (N222, N264 and N272) all lie within the dimerisation domain, we wished to determine whether their mutation affected dimer stability, and identified N264 as an amino acid that plays a critical role in dimer stability. Our attempts to restore dimer stability by substituting amino acids other than glutamine at this position in CPG*(Q)3 gave mixed results. Mutation to serine restored dimer stability and doubled enzyme activity, though with some cost to Km. In contrast, substitution with threonine did not stabilise the dimers, and substitution with alanine gave a practically inactive enzyme.

We were intrigued to note that the surface tethering process appeared to overcome many of the detrimental effects induced by the N264 mutations. Thus the Km values for cytosolic CPG2* proteins in which N222 and N272 were substituted with glutamine, but position 264 was substituted for glutamine, alanine, serineor threonine, fell in the range of approximately 10 μm to 125 μm (unmeasurable for CPG2* (QAQ)). The corresponding surface tethered enzymes had a more narrow range of Km values, from 49 to 69 μm. Similarly, whereas the cytosolic proteins had catalytic values that were from 78 to 32% (unmeasurable for CPG2* (QAQ)) of the activity of CPG2*, the surface tethered proteins had activities ranging form 29 to 40% of the activity of CPG2*. Thus the mutations at position 264 had more significant effects on enzyme activity in the soluble, cytosolic enzymes than in the membrane tethered proteins.

Despite the problems associated with glycosylation and the mutations that were made to prevent it, CPG2 is an ideal enzyme for the surface tethered approach. It is normally secreted into the periplasmic space and since it does not require any cytosolic factors for activity it can be expressed in an active, surface tethered form provided that glycosylation is prevented. Furthermore, CPG2 does not require additional metabolic steps to generate the toxic moiety from CMDA, because it releases the drug directly, and since the drug is lipophilic, it can enter all cells without the need of active transport to mediate cell death.

One potential disadvantage of the surface tethered protein was a generally higher km for substrate compare with internally expressed protein. Thus CPG2*(Q)3 had Km values of 7-10 μm, whereas all the surface tethered proteins had Km values that were 4-6 fold higher. However, in the presence of excess prodrug a reduced affinity for prodrug (high km) may be of little importance. In an ADEPT trial, peak CMDA concentrations of approximately 3mM have been measured in the sera of patients, demonstrating that very high concentrations of prodrug can be achieved.

Ultimately, the surface tethered protein was highly active, with a catalytic value that was 40% of that of the wild-type protein and yet we consistently observed concentrations of CPG2 enzyme activity in the stCPG2(Q)3 expressing clones that were significantly lower than the 40% expecte when compared to corresponding CPG2* expressing cells. This was most obvious with SK-OV-3 cells, where the stCPG2(Q)3 expressing clones expressed only 2% of the CPG2 activity of the corresponding CPG2* expressing cells, yet gave similar cytoxicity and bystander effects. Even with WiDr cells, which had the least difference, the stCPG2(Q)3 clones expressed only 10% of the activity of the CPG2* expressing cells. This suggests that the stCPG2(Q)3 protein cannot be expressed at, or accumulate to the high levels that can be achieved by CPG2*.

TABLE 1
Kinetic parameters of CPG2-derived enzymes

| Cell extract | Kₘ (μM MTX) | Catalytic activity (%) |
|---|---|---|
| COS/CPG2* | 7.2 | 100 |
| COS/CPG2* (Q) 3 | 10.4 | 32 |
| COS/CPG2* (QSQ) | 58 | 58 |
| COS/CPG2* (QAQ) | na | Very low |
| COS/CPG2* (QTQ) | 125 | 78 |
| COS/stCPG2 (Q) 3 | 54 | 40 |
| COS/stCPG2 (QSQ) | 51 | 40 |
| COS/stCPG2 (QAQ) | 69 | 38 |
| COS/stCPG2 (QTQ) | 45 | 29 |
| COS/stCPG2 (Q, NWV, Q) | 90 | 55 |
| WiDr/stCPG2 (Q) 3 (W3) | 38 | 41 |
| WiDr/stCPG2 (Q) 3 (W4) | 40 | 46 |

Na: not applicable,
nd: not determined

TABLE 2
Cytotoxicity of CMDA to cell lines expressing
stCPG2 (Q) 3 (WiDr, SK-OV-3 and A2780) and
expressing CPG2* (SK-OV-3).

| | Clone and | | | Differential | CPG2 |
|---|---|---|---|---|---|
| Tumor | protein | IC₅₀ (μM | | cytotoxicity | activity |
| cell line | expressed | CMDA) | | (fold) | (U/mg)^{a} |
| WiDr | W3 stCPG2 (Q) 3 | 100 | (±10) | 32.3 | 0.177 |
| | W4 stCPG2 (Q) 3^{b} | 147 | (±10) | 22.0 | 0.226 |
| | lacZ^{c} | 3230 | (±120) | na | 0 |
| SK-OV-3 | S5 stCPG2 (Q) 3^{b} | 216 | (±31) | 19.4 | 0.023 |
| | S6 stCPG2 (Q) 3 | 479 | (±22) | 8.7 | 0.020 |
| | S7 stCPG2 (Q) 3 | 544 | (±29) | 7.7 | 0.016 |
| | lacZ^{c} | 4180 | (±452) | na | 0 |
| | S2.34 CPG2* | 1289 | (±221) | 3.2 | 0.023 |
| | S2.4 CPG2* | 3196 | (±166) | 1.3 | 0.014 |
| A2780 | A3 stCPG2 (Q) 3 | 15.6 | (±1.5) | 138 | 0.067 |
| | A4 stCPG2 (Q) 3^{b} | 20.4 | (±0.6) | 105 | 0.060 |
| | lacZ^{c} | 2150 | (±180) | na | 0 |

^{a}U/mg detergent soluble protein.
^{b}cell lines selected for bystander cytotoxicity and time-course toxicity analyses.
^{c}cell lines expressing lacZ, characterised in reference 30.

1. A bacterial carboxypeptidase enzyme which, in its native form, comprises one or more asparagine residues, the residues being part of motifs which on expression in a mammalian cell are subject to N-linked glycosylation, wherein at least one asparagine residue is altered to serine, and which enzyme retains carboxypeptidase activity.

2. A bacterial carboxypeptidase enzyme according to claim 1, which enzyme, in its native form, comprises three asparagine residues; Asn (1), Asn (2) and Asn (3) numbered in the N-terminal to C-terminal direction, the residues being part of motifs which on expression in a mammalian cell are subject to N-linked glycosylation, wherein Asn (2) is altered to serine.

3. A bacterial carboxypeptidase enzyme according to claim 2 wherein Asn (1) and Asn (3) are altered to glutamine.

4. A bacterial carboxypeptidase enzyme according to any one of the preceding claims which is the bacterial carboxypeptidase enzyme CPG2.

5. A bacterial carboxypeptidase enzyme according to claim 4, which in its native form is obtainable from a Pseudomonas.

6. A bacterial carboxypeptidase enzyme CPG2 having has the amino acid sequence shown in SEQ ID NO:2, which is altered according to any one of the preceding claims.

7. A bacterial carboxypeptidase enzyme according to claim 6 wherein Asn 264 is altered to serine.

8. A bacterial carboxypeptidase enzyme according to claim 7 wherein Asn 264 is altered to threonine.

9. A bacterial carboxypeptidase enzyme according to claim 7 or claim 8, wherein Asn 264 and Asn 272 are altered to glutamine.

10. A bacterial carboxypeptidase which is a mutant, variant, homologue, or allele of the enzyme shown in SEQ ID NO:2, which is altered according to any one of the preceding claims.

11. A vector comprising a nucleic acid sequence encoding the carboxypeptidase of any one of the preceding claims.

12. A vector according to claim 11 which further comprises a signal sequence capable of directing expression of the carboxypeptidase to the surface of a mammalian cell.

13. A vector according to claim 12 wherein the signal sequence is a signal peptide of a transmembrane receptor kinase.

14. A two component system for use in association with one another comprising:
(a) a vector capable of expressing an enzyme according to any one of claims 1 to 10; and
(b) a prodrug which can be converted into an active drug by said enzyme.

15. A system according to claim 14 wherein the prodrug is a nitrogen mustard prodrug.

16. A system according to claim 14 or claim 15 wherein the vector comprises a signal peptide capable of targetting the carboxypeptidase to the surface of a mammalian cell.

17. A system according to claim 16 wherein the signal sequence is a signal peptide of a transmembrane receptor kinase.

18. A system according to any one of claims 14 to 17 wherein the vector comprises a promoter capable of being expressed in a tissue restricted manner.

19. A system according to claim 18 wherein the promoter is c-erbB2 promoter.

20. A two component system for use in association with one another comprising:
(a) a tumour specific micro-organism, comprising a vector capable of expressing an enzyme according to any one of claims 1 to 10; and
(b) a prodrug which can be converted into an active drug by said enzyme.

21. A system according to any of claims 14 to 20 or a carboxypeptidase according to any one of claims 1 to 13 for use in a method of treatment or therapy of the human or animal body.

22. A method of removing a moiety —NH—CH(CO2H) (Z) from a compound to which the moiety is attached via an amide linkage, the method comprising contacting a said compound with an enzyme according to any one of claims 1 to 14 release said moiety.


SEQUENCE LISTING (6 sequences)

SEQ ID NO: 1 - DNA, 2048 bp, Pseudomonas sp.
  CDS (195)..(1442)
atcatggatccacgcactgaaggcgcgcggcaagacgcgcggcgtggcgacgctgtgcatcggcgggggcgaaggcaccgcagtggcactcgaattgctataagaaccatggctggggacgcccgacaacaggcgtccaccagcttttttcattccgacaacccgaacgaacaatgcgtagagcaggagattccatgcgcccatccatccaccgcacagccatcgccgccgtgctggccaccgccttcgtggcgggcaccgccctggcccagaagcgcgacaacgtgctgttccaggcagctaccgacgagcagccggccgtgatcaagacgctggagaagctggtcaacatcgagaccggcaccggtgacgccgagggcatcgccgctgcgggcaacttcctcgaggccgagctcaagaacctcggcttcacggtcacgcgaagcaagtcggccggcctggtggtgggcgacaacatcgtgggcaagatcaagggccgcggcggcaagaacctgctgctgatgtcgcacatggacaccgtctacctcaagggcattctcgcgaaggccccgttccgcgtcgaaggcgacaaggcctacggcccgggcatcgccgacgacaagggcggcaacgcggtcatcctgcacacgctcaagctgctgaaggaatacggcgtgcgcgactacggcaccatcaccgtgctgttcaacaccgacgaggaaaagggttccttcggctcgcgcgacctgatccaggaagaagccaagctggccgactacgtgctctccttcgagcccaccagcgcaggcgacgaaaaactctcgctgggcacctcgggcatcgcctacgtgcaggtcaacatcaccggcaaggcctcgcatgccggcgccgcgcccgagctgggcgtgaacgcgctggtcgaggcttccgacctcgtgctgcgcacgatgaacatcgacgacaaggcgaagaacctgcgcttcaactggaccatcgccaaggccggcaacgtctcgaacatcatccccgccagcgccacgctgaacgccgacgtgcgctacgcgcgcaacgaggacttcgacgccgccatgaagacgctggaagagcgcgcgcagcagaagaagctgcccgaggccgacgtgaaggtgatcgtcacgcgcggccgcccggccttcaatgccggcgaaggcggcaagaagctggtcgacaaggcggtggcctactacaaggaagccggcggcacgctgggcgtggaagagcgcaccggcggcggcaccgacgcggcctacgccgcgctctcaggcaagccagtgatcgagagcctgggcctgccgggcttcggctaccacagcgacaaggccgagtacgtggacatcagcgcgattccgcgccgcctgtacatggctgcgcgcctgatcatggatctgggcgccggcaagtgaatgctgccccccggcttttcactcgcgttgctcgtgtaactccaccccccgagggggaggcgcggtccgccttggggcggcccggcggcgaccgcctcgtcacatagaaggaactgccatgttgttgacagcagaccaggaagccatccgcgacgcggtgcgcgacttctcgcaagccgaactctggcccaacgccgcgaatggggaccgcgagcacagctttcccaagagcccaccaggccgtcggctggcgtacgcagtctgcgtgcccgaggagcatggcggcgccggcctcgactacctcacctcgcgctggtgctggaggagatcgcggccggcgacggcggcaccagcaccgccatcagcgtgaccaactgccccgtcaacgccatcctcatgcgctacggcaacgcgcagcagaagaagcagtggctcgagccgctggcgcagggccggatgctcggcgccttctgcctgaccgagccgcaggccggcagcgatgcatcgagcctgcgcaccacggcgcgcaaggacggcgacggctacgtgatcgacggcgtgaagcagttcatcaccagcggcaagaacggccaggtggcgggatcc

SEQ ID NO: 2 - protein, 415 aa, Pseudomonas sp.
MRPSIHRTAIAAVLATAFVAGTALAQKRDNVLFQAATDEQPAVIKTLEKLVNIETGTGDAEGIAAAGNFLEAELKNLGFTVTRSKSAGLVVGDNIVGKIKGRGGKNLLLMSHMDTVYLKGILAKAPFRVEGDKAYGPGIADDKGGNAVILHTLKLLKEYGVRDYGTITVLFNTDEEKGSFGSRDLIQEEAKLADYVLSFEPTSAGDEKLSLGTSGIAYVQVNITGKASHAGAAPELGVNALVEASDLVLRTMNIDDKAKNLRFNWTIAKAGNVSNIIPASATLNADVRYARNEDFDAAMKTLEERAQQKKLPEADVKVIVTRGRPAFNAGEGGKKLVDKAVAYYKEAGGTLGVEERTGGGTDAAYAALSGKPVIESLGLPGFGYHSDKAEYVDISAIPRRLYMAARLIMDLGAGK

SEQ ID NO: 3 - DNA, 42 bp, Artificial Sequence
  Description of Artificial Sequence Oligonucleotide
cgccaaggccggccaagtctcggggacaacatcatccccgcc

SEQ ID NO: 4 - DNA, 42 bp, Artificial Sequence
  Description of Artificial Sequence Oligonucleotide
ggcggggatgatgttgtccccgagacttggccggccttggcg

SEQ ID NO: 5 - DNA, 36 bp, Artificial Sequence
  Description of Artificial Sequence Oligonucleotide
aagaaacctgcgcttcgtccccaatggaccatcgcc

SEQ ID NO: 6 - DNA, 35 bp, Artificial Sequence
  Description of Artificial Sequence Oligonucleotide
ggcgatggtccattggggacgaagcgcaggttctt